(19) United States
(12) Patent Application Publication
(10) Pub. No.: US 2021/0128849 A1
(43) Pub. Date: May 6, 2021

(54) NASAL CANNULA AND TUBING MEDICINE DELIVERY SYSTEM

(71) Applicant: Neotech Products LLC, Valencia, CA (US)

(72) Inventors: Matthew McNally, Sunapee, NH (US); Richard Mitchell, Fairlee, VT (US); Lawrence Kiliszewski, The Woodlands, TX (US); Craig McCrary, Valencia, CA (US)

(73) Assignee: Neotech Products LLC, Tokyo (JP)

(21) Appl. No.: 17/150,692

(22) Filed: Jan. 15, 2021

Related U.S. Application Data
(63) Continuation-in-part of application No. 16/436,615, filed on Jun. 10, 2019.
(60) Provisional application No. 63/075,239, filed on Sep. 7, 2020.

(51) Int. Cl. A61M 15/08

(52) U.S. Cl. CPC A61M 15/08

(57) ABSTRACT

A nasal cannula system having nasal prongs with an ancillary delivery system for a treatment fluid into a patient wearing the nasal cannula without removing the cannula. The nasal prongs have parallel ports that fluidly connect the delivery system to the nasal ports on the nasal prongs. The delivery system provides the treatment fluid in an aerosolized form. This configuration combines the aerosolized treatment fluid with the air or other fluid ordinarily delivered by the nasal cannula. The system may also use airway tubes having a generally semi-circular cross-section with an internal rib extending the length of the tube. The semi-circular cross-section provides a flat exterior surface for abutment or contact with a patient's skin and the rib prevents pinching closure of the airway tube.

### RELATED APPLICATIONS

This application is a continuation-in-part of U.S. application Ser. No. 16/436,615, filed Jun. 10, 2019. This application also claims the benefit of U.S. Provisional Application No. 63/075,239, filed on Sep. 7, 2020.

### BACKGROUND OF THE INVENTION

The present invention is directed to airway delivery systems used in medical treatment, and more particularly to tubing and connections for the system having a specific shape and configuration to facilitate delivery of medicine to patients. The shape of the tubing and connections is intended to provide a more comfortable and stable placement of the airway delivery systems on and around a patient, particularly their face. The configuration to facilitate delivery of medicine is to provide a convenient way of delivering aerosolized medicine to patients.

Nasal continuous positive airway pressure, NCPAP, is a standard used for administration of non-invasive positive airway pressure, particularly in the Neonate. Administration of non-invasive positive airway pressure is usually accomplished by tubing being run from behind or above a patient's head to their nose where cannula are inserted into the nasal openings. The tubing is generally run above a patient's ears and across the cheeks.

Typical tubing and connections for such systems have a circular cross-section. One drawback of tubing and connections with a circular cross-section is that the same can easily slide and/or roll relative to the face or head of the patient. When the tubing and connections slide and/or roll, such can tend to become dislodged from the nasal openings and cease to provide airflow. Another drawback is that such circular tubing can create pinch and pressure points when a patient may lay on the tubing. Such drawbacks can be exacerbated when the systems are used in neonatal settings. Infant patients cannot be given direction or instruction to not move and avoid dislodging or pinching the tubing and connections.

In addition, it is sometimes advantageous to deliver medicine to a patient in aerosolized form. Such delivery may be done through oral or nasal openings, but such administration can be hindered by CPAP or NCPAP devices being worn by patients. Accessing nasal or oral openings while a patient wears a device can hinder the delivery of aerosolized medicines, particularly that does not require the removal of CPAP or similar devices.

Accordingly, there is a need for tubing and connections for use in airway delivery systems that minimize the risk of sliding, moving, dislodging, and pinching through patient movement during use, particularly in neonatal patients. The present invention fulfills these needs and provides other related advantages.

### SUMMARY OF THE INVENTION

The present invention is directed to a nasal cannula system, primarily for use in the delivery of fluids in a medical setting. The nasal cannula system includes a nasal body having a nare tube with a nare port configured for insertion into a nasal passage when worn by a patient. The nare tube is in fluid communication with an internal passage in the nasal body. The nare tube also has an external parallel port that is in fluid communication with the nare port. An ancillary delivery apparatus having an inlet tube is connected to the external parallel port.

In a particularly preferred embodiment, the nasal body includes a pair of nare tubes, both in fluid communication with the internal passage. In this embodiment, each of the pair of nare tubes has an external parallel port. The ancillary delivery apparatus preferably has a pair of inlet tubes, where each of the pair of inlet tubes is connected to one of the external parallel ports on the pair of nare tubes. An air hose is preferably fluidly connected to the pair of inlet tubes.

In a further embodiment, the ancillary delivery apparatus has an aerosolizer for combining an ancillary material with an external air flow, as from the air hose. A hopper on the ancillary delivery apparatus preferably contains the ancillary material and introduces the ancillary material to the aerosolizer.

The nasal cannula system may further include an elongated gas delivery tube communicatingly connected to the nasal body. The gas delivery tube has a semi-circular cross-section with an internal rib extending along a length of the gas delivery tube. The internal rib may be continuous along the length of the gas delivery tube so as to divide the gas delivery tube into two separate passageways. Alternatively, the internal rib may be discontinuous along the length of the gas delivery tube so as to divide the gas delivery tube into two passageways that are in fluid communication through discontinuities in the internal rib. The gas delivery tube preferably has a generally flat exterior side configured to lay against a patient's skin when worn.

In a particularly preferred embodiment, the nasal cannula system has a nasal prong with a pair of nare tubes, each ending in a nare port configured for insertion into a nasal passage when worn by a patient. The pair of nare tubes are in fluid communication with an internal passage in the nasal prong. Each of the pair of nare tubes includes an external parallel port that is in fluid communication with the nare port. The ancillary delivery apparatus has a pair of inlet tubes, each one connected to the external parallel port on one of the pair of nare tubes.

The elongated gas delivery tube may be communicatingly connected to the nasal body to provide fluid flow to the internal passage and the nasal prongs in the nasal cannula system. In a particularly preferred embodiment, the gas delivery tube has in cross-section a generally flat side joined to a generally curved side that together form a passageway therebetween having a semi-circular cross-section. An internal rib extends through the passageway along a length of the gas delivery tube, spanning from the flat side to the curved side.

In a particularly preferred embodiment, the gas delivery tube is a pair of gas delivery tubes, each of said pair of gas delivery tubes configured to lay against opposite sides of a patient's head when worn. The system further has a supply adapter with a pair of gas ports each having a semi-circular cross-section matching that of the passageway of one of the pair of gas delivery tubes. Alternatively, the supply adapter may have a connection port with a generally circular cross-section configured to receive the pair of gas delivery tubes with flat exterior sides abutting. Still alternatively, the supply adapter may have a pair of connection ports, each of the pair of connection ports having a semi-circular cross-section matching that of one of the pair of gas delivery tubes.

Other features and advantages of the present invention will become apparent from the following more detailed description, taken in conjunction with the accompanying drawings, which illustrate, by way of example, the principles of the invention.

### BRIEF DESCRIPTION OF THE DRAWINGS

The accompanying drawings illustrate the invention. In such drawings:

FIG. 1 is an environmental view illustrating an application of a first preferred embodiment of the inventive nasal cannula system as attached to an infant patient;

FIG. 2 is a perspective view of the first preferred embodiment of the inventive nasal cannula system;

FIG. 3 is a close-up perspective view of the connection between a tube and a connector of a preferred embodiment of the inventive nasal cannula system;

FIG. 4 is a perspective view of the inventive nasal cannula system;

FIG. 4A is a partial cut-away of a first preferred embodiment of the airway tube of the inventive nasal cannula system;

FIG. 4B is a partial cut-away of a second preferred embodiment of the airway tube of the inventive nasal cannula system;

FIG. 4C is a partial cut-away of a third preferred embodiment of the airway tube of the inventive nasal cannula system;

FIG. 5 is a perspective view of a length of a first preferred embodiment of the inventive gas supply tube;

FIG. 6 is a perspective view of a length of a second preferred embodiment of the inventive gas supply tube;

FIG. 7A is a perspective view of a length of gas supply tube being inserted into a supply adapter;

FIG. 7B is a cut-away perspective view of a length of gas supply tube being inserted into a supply adapter;

FIG. 8 is an illustration of the application of the inventive nasal cannula system to a patient;

FIG. 9 is an illustration of the connection of the inventive nasal cannula system to gas supply tubing with separate inhalation and exhalation supplies;

FIG. 10 is a perspective view of the inventive nasal cannula system with ancillary delivery tubing;

FIG. 11 is a close-up view of the nasal body and ancillary delivery tubing of FIG. 10;

FIG. 11A is a partially exploded view of the inventive nasal cannula and ancillary delivery tubing;

FIG. 11B is a partial cross-sectional view of the nasal body, nasal prongs, and parallel ports of the inventive nasal cannula system;

FIG. 12 is an illustration of the application of the inventive nasal cannula system with ancillary delivery tubing to a patient;

FIG. 13 is an illustration of the connection of the inventive nasal cannula system with ancillary delivery tubing to gas supply tubing with a single inhalation supply; and

FIG. 14 is an illustration of the inventive nasal cannula system with ancillary delivery tubing and a medicine aerosolizer.

### DETAILED DESCRIPTION OF THE PREFERRED EMBODIMENTS

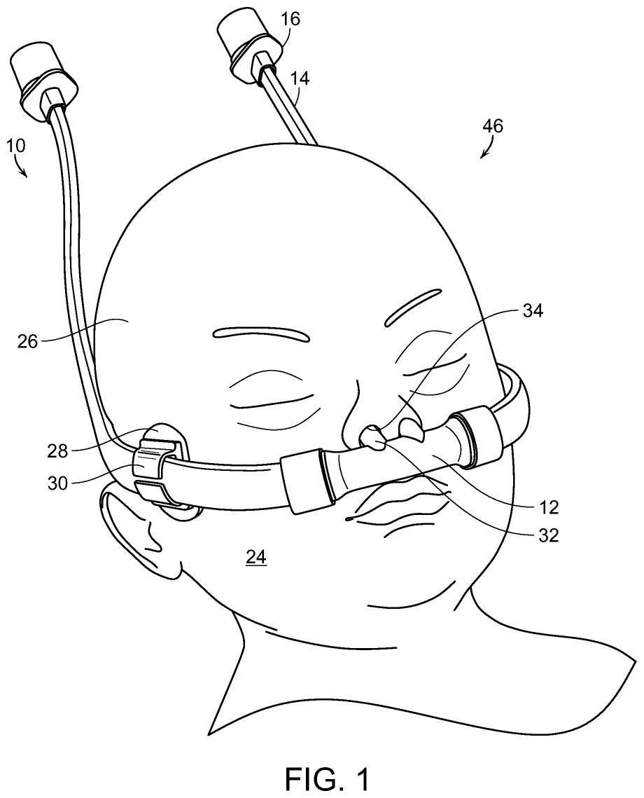

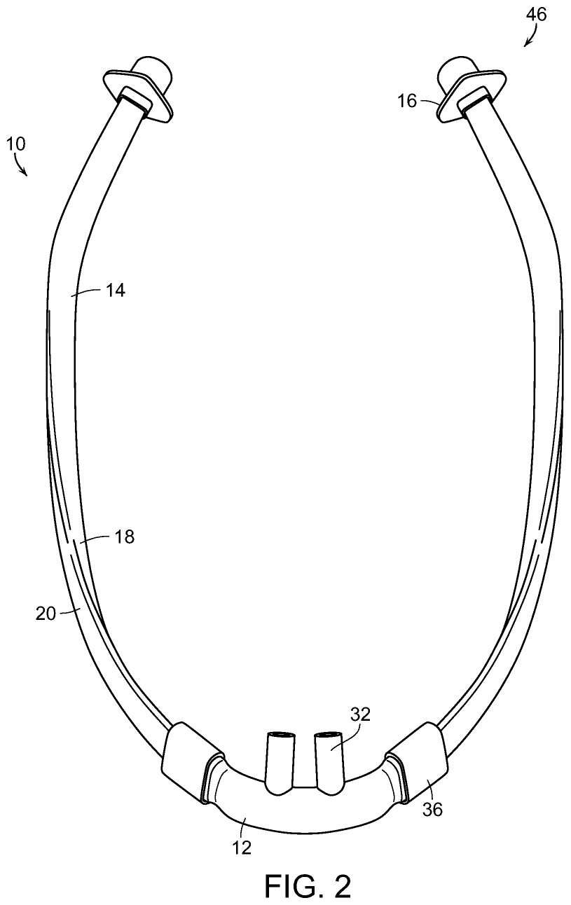

In the following detailed description, the nasal cannula system of the present invention is generally referred to by reference numeral 10 in FIGS. 1-4. The primary components of the system 10 represent the nasal body 12, the airway tubing 14, and the supply adapter 16.

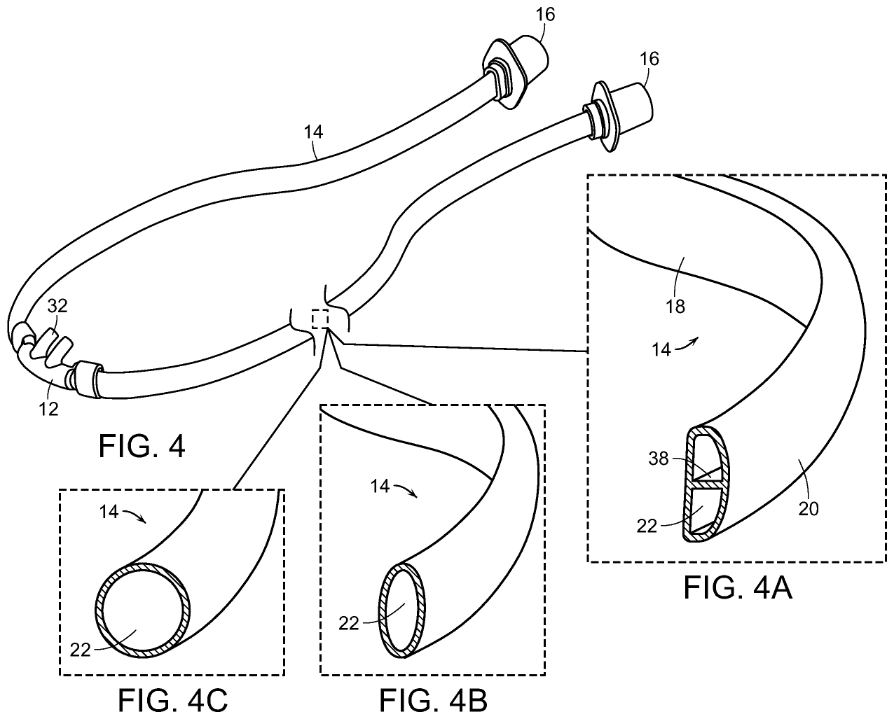

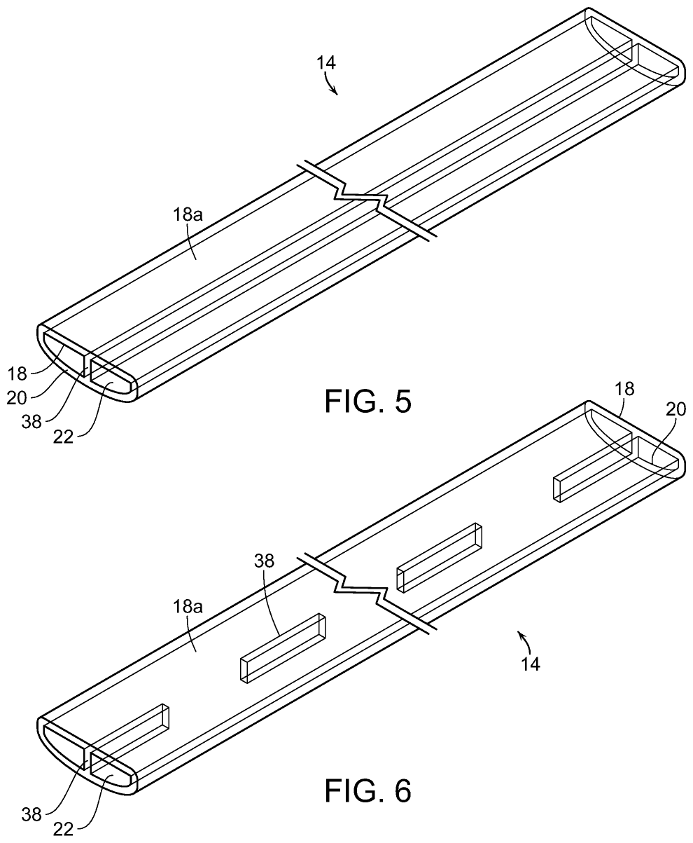

The airway tubing 14, shown in cross-section in FIG. 4A and in transparent form in FIGS. 5 and 6, preferably has a generally semi-circular or D-shaped cross-section. The airway tubing 14 may also come in other cross-sectional shapes. FIG. 4B shows an alternate embodiment for the airway tubing 14 that has an oval cross-section. FIG. 4C shows another alternate embodiment for the airway tubing 14 that has an oval cross-section. FIG. 4D shows another alternate embodiment for the airway tubing 14 that has a circular cross-section. Such oval cross-section presents similar benefits to those of the semi-circular cross-section described below.

The semi-circular cross-sectional tubing 14 has a generally flat side 18 and a generally curved side 20 that a connected to form a passageway 22 having the semi-circular cross-section. The flat side 18 is flat on at least an exterior surface 18a relative to the tubing 14, but is preferably flat on an interior surface as well. Similarly, the curved side 20 is curved on at least an exterior surface 20a relative to the tubing 14, but is preferably curved on an interior surface as well.

When the flat side 18 and curved side 20 are combined, the exterior surfaces 18a, 20a combined to create a generally semi-circular cross-section on the exterior of the tubing 14. Similarly, when the interior surfaces of the flat side 18 and curved side 20 are flat and curved, respectively, they form a passageway 22 through the tubing 14 that has a semi-circular cross-section.

The flat exterior surface 18a of the flat side 18 is configured to lay flush against the skin of a patient when the tubing 14 is used. In this way, the tubing 14 has a lower profile when resting against the skin 24 of a patient 26, e.g., on the cheek or otherwise around the face or head, as shown in FIG. 1. This lower profile minimizes the degree to which a patient, particularly an unconscious or an infant patient, may disturb or dislodge the tubing 14 or system 10 due to interference from hand or arm movement near the tubing 14 or contact with other objects, e.g., pillows, due to movement of the head. The lower profile and exposed exterior curved surface 20a also minimizes pinching or pressure points when the tubing 14 may be pressed between the patient's skin and an external object, i.e., a pillow or other medical device.

The flat exterior surface 18a also minimizes the degree to which the inventive tubing 14 may roll, slide, or otherwise be displaced when in use on a patient. The flat exterior surface 18a provides a stable surface with increased contact against the skin 24 of a patient. Such stable surface minimizes rolling and the increased contact minimizes sliding or other movement across the skin 24. To assist in this effort, the system 10 may include a surface retainer 28 that is configured to removably adhere to the skin 24 of a patient 26. The surface retainer has a clamp portion 30 is slightly raised above the skin 24 and has a semi-circular cross-section that generally matches the semi-circular cross-section of the tubing 14.

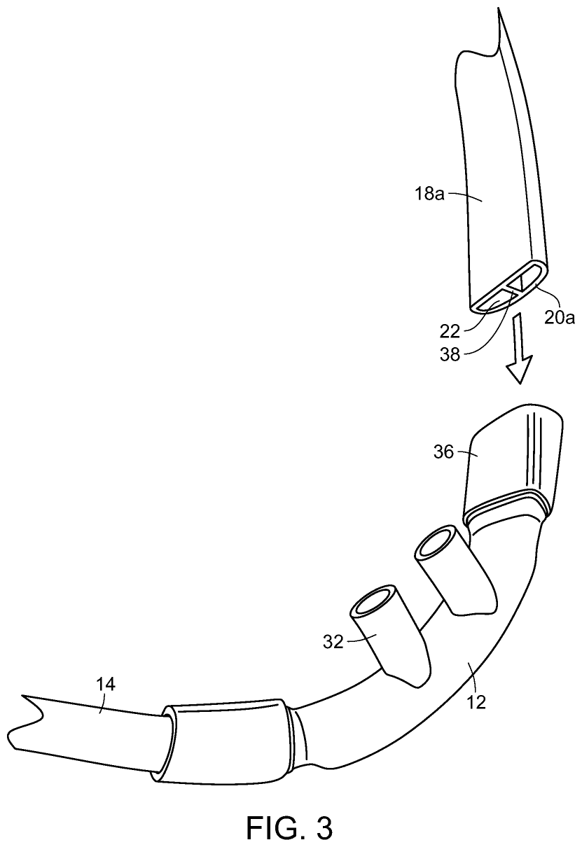

As shown in FIG. 3, the nasal body 12 comprises an elongated tube and preferably has a flat exterior surface 12a that is positioned so as to lay flush against the patient's skin 14, e.g., on the upper lip beneath the nose. The nasal body 12 includes a pair of nasal prongs 32 that are configured to enter nasal openings 34 when in use. The nasal body 12 also includes at least one tube connector 36 at one end of the body 12 for receipt of airway tubing 14. The tube connector 36 also has a generally flat exterior surface 36a that matches the flat exterior surface 12a of the nasal body 12. The tube connector 36 also preferably has a semi-circular cross-section that matches the semi-circular cross-section of the airway tubing 14. Where the airway tubing 14 has a shape other than semi-circular, the tube connectors 36 have a cross-section to match the airway tubing 14.

Where the nasal body 12 has a single tube connector 36 at one end, the other end of the nasal body is closed off. In this way, airway tubing 14 can introduce oxygen or another gas into the nasal body 12 for passage through the nasal prongs 32 into the nasal openings 34. Preferably, the nasal body 12 has tube connectors 36 opposite ends thereof, each having a generally semi-circular cross-section configured for sliding reception of inventive airway tubing 14. In this way, oxygen or other gases can be introduced into the nasal body 12 for administration to the patient 26. The airway tubing 14 may include an inhalation tube 14a wherein fluid flows toward the nasal body 12 and an exhalation tube 14b wherein fluid flows away from the nasal body 12.

The tubing 14 preferably has an internal rib 38 that runs the length of the tubing 14 through the passageway 22. The internal rib 38 is designed to provide additional rigidity to the tubing 14 such that the passageway 22 does not become completely closed off or otherwise blocked when the patient 26 may lay on the tubing 14 or other object exerts pressure on the exterior of the tubing 14. The internal rib 38 may be continuous so as to completely divide the passageway 22 into two separate passageways.

Alternatively, and preferably, the internal rib 38 may be discontinuous so as not to completely divide the passageway 22. Periodic gaps in the internal rib 38 allow for all gases to flow to the other side of the discontinuous internal rib 38 when one side of the passageway 22 may become pinched or blocked. In tubing 14 that has a continuous internal wall 38, pinching or blocking of one side of the passageway 22a or 22b may lead to uneven pressure distribution of the gas causing improper administration.

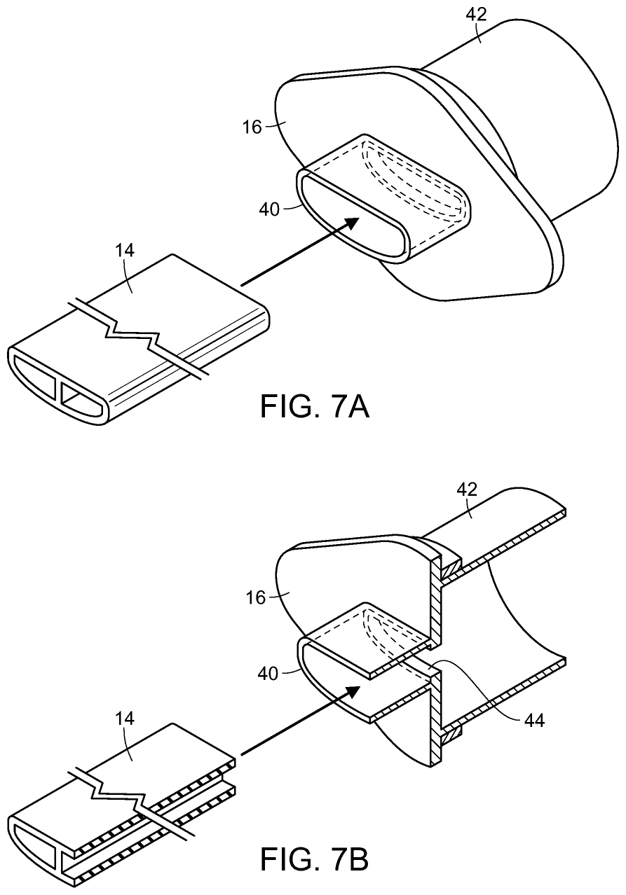

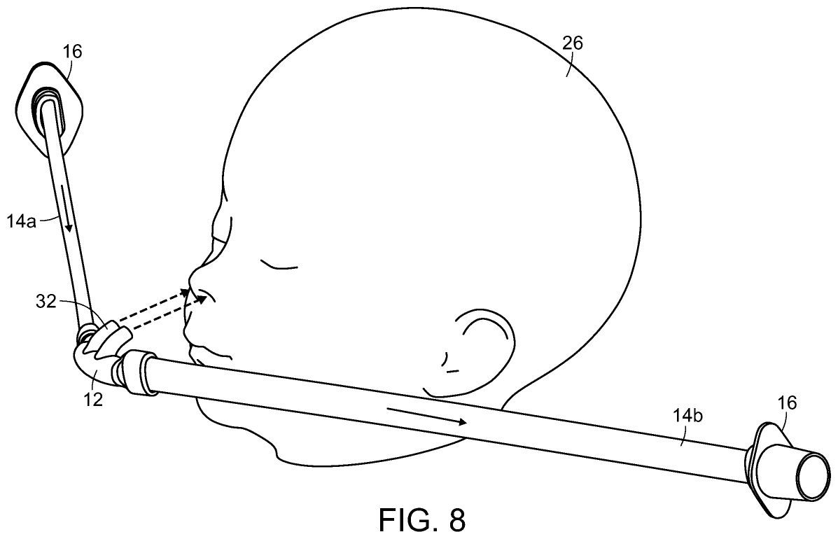

The supply adapter 16, various embodiments shown in detail in FIGS. 7A-7B, generally comprises a connector or coupling with a tube port 40 and a supply port 42 oppositely disposed. The tube port 40 preferably has a semi-circular cross-section that matches the semi-circular cross-section of the airway tubing 14. An opening 44 fluidly connects the tube port 40 to the supply port 42. Preferably, the opening 44 also has a semi-circular shape to match the shape of the tube port 40 and the airway tube 14. The supply port 42 should have a shape to match the connection available on gas supply tube 50 as may be found in a hospital or medical facility as from a ventilator or similar machine, to which the supply port 42 is configured to connect.

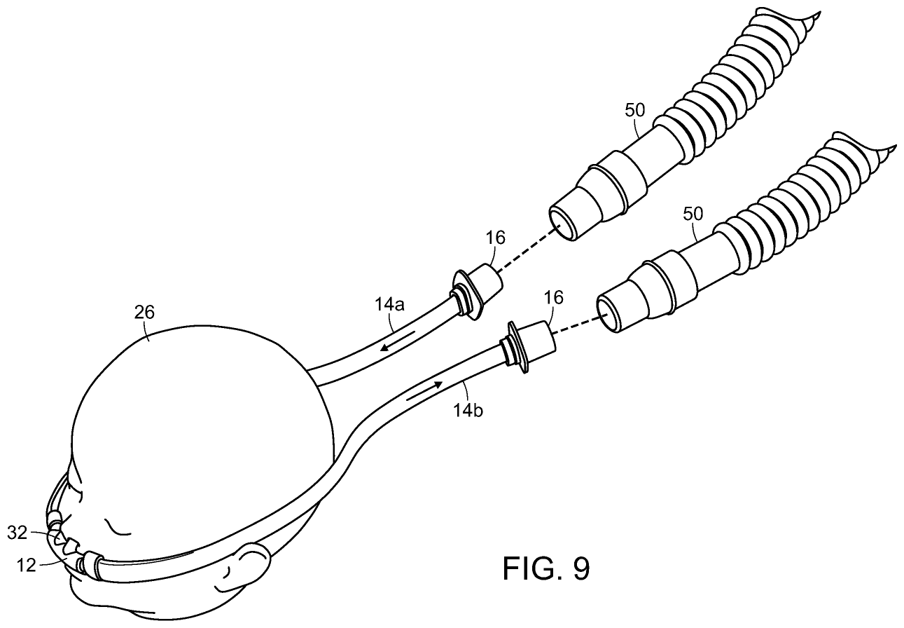

This embodiment of supply adapter 16 may be used in the system 10 in multiple configurations. A single supply adapter 16 can be connected to a single length of airway tubing 14 that is in turn connected to a nasal body that is closed at the opposite end. The single supply adapter 16 is then connected to a gas supply that provides gas for administration to a patient. Alternatively, two supply adapters 16 can be separately connected to different lengths of airway tubing 14, which separate lengths of airway tubing 14 are in turn connected to opposite ends of a nasal body 12. Each separate supply adapter 16 is connected to appropriately positioned gas supply connections to provide gas for administration to a patient. This configuration of two separate supply adapters 16, as shown in FIGS. 1 and 9, is referred to as an open-ended configuration 46.

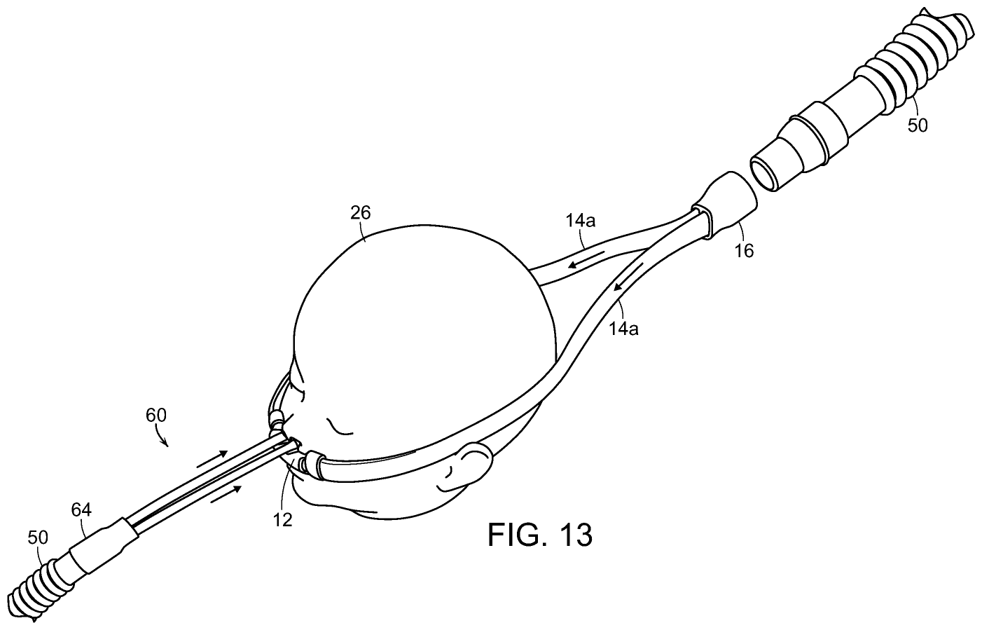

In an alternate embodiment, the tube port 40 on the supply adapter 16 may comprise two tube ports 40 for receiving two separate airway tubes 14 into the same supply adapter 16. In this embodiment, each of the two tube ports 40 may each have separate openings 44 connecting the tube ports 40 to the same supply port 42. The supply port 42 is the same shape as the earlier embodiment and configured for connection to a gas supply. In this embodiment, separate lengths of airway tubing 14 are connected to each of the tube ports 40 on the same supply adapter 16 with opposite ends of the airway tubes 14 connected to the a nasal body 12. The single supply adapter 16 is connected to an appropriately positioned gas supply tube 50 to provide gas for administration to a patient. In this configuration, both airway tubes as inhalation tubes 14a as fluid in both tubes flow from the adapter 16 toward the nasal body 12, as shown below in FIG. 13, which is referred to as a close-ended configuration 52.

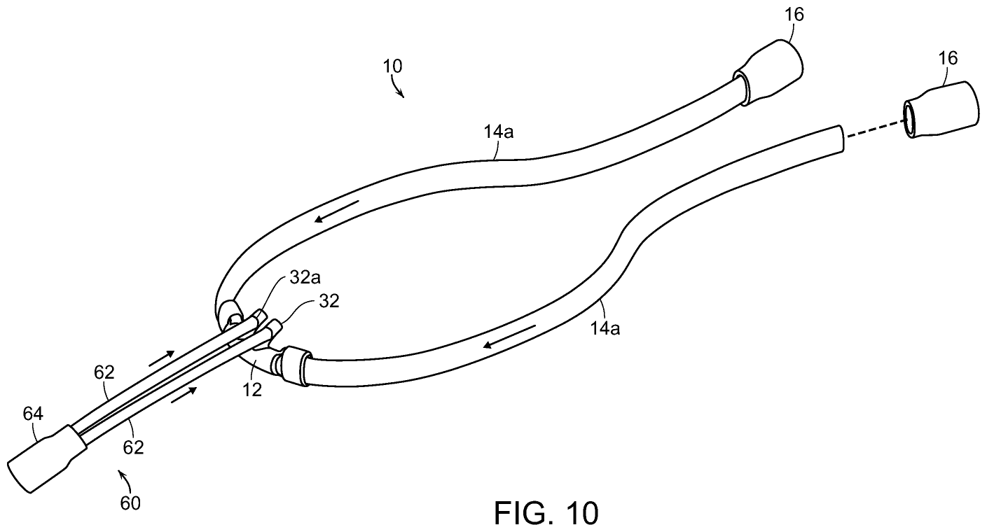

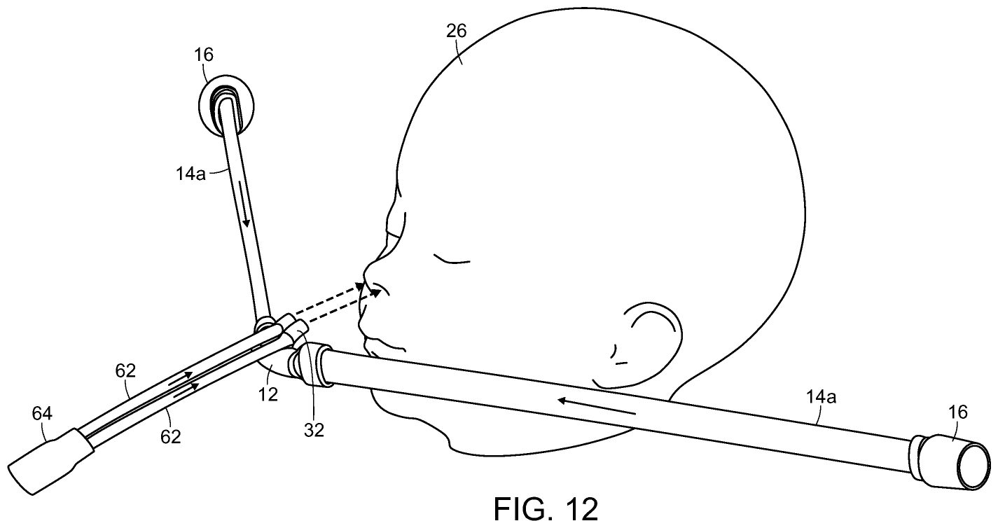

In a particular preferred embodiment, shown in FIGS. 10-14B, the nasal cannula system 10 includes an ancillary delivery system 60 in fluid communication with the nasal prongs 32. In this embodiment including the ancillary delivery system 60, the nasal cannula system 10 may be constructed wherein the airway tubing 14 has any cross-section, i.e., circular, oval, or semi-circular (D-shaped), as described above. The ancillary delivery system 60 comprises two inlet tubes 62 that are connected at one end to parallel ports 32a on the nasal prongs 32 and connected at an opposite end to a common coupling 64. The parallel ports 32a are in fluid communication with the interior of the nasal prongs 32 such that any fluid introduced through the ancillary delivery system 60 is mixed with any fluid flowing through the nasal cannula system 10.

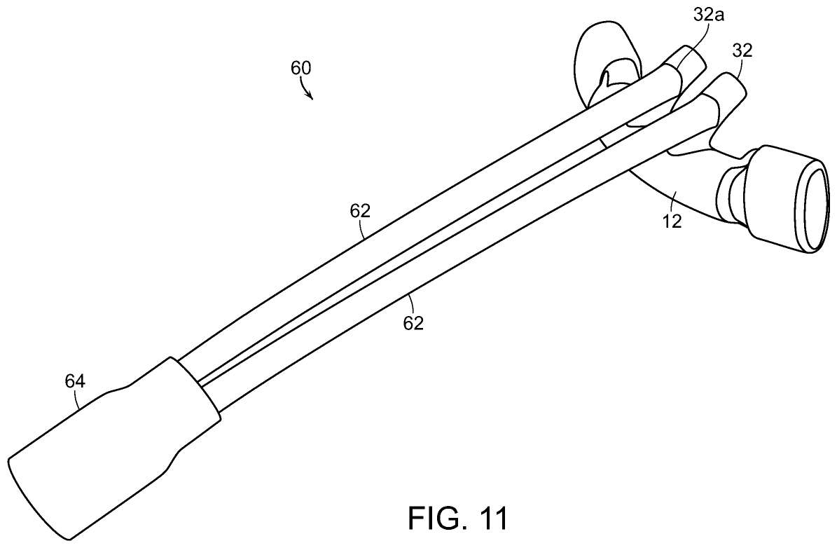

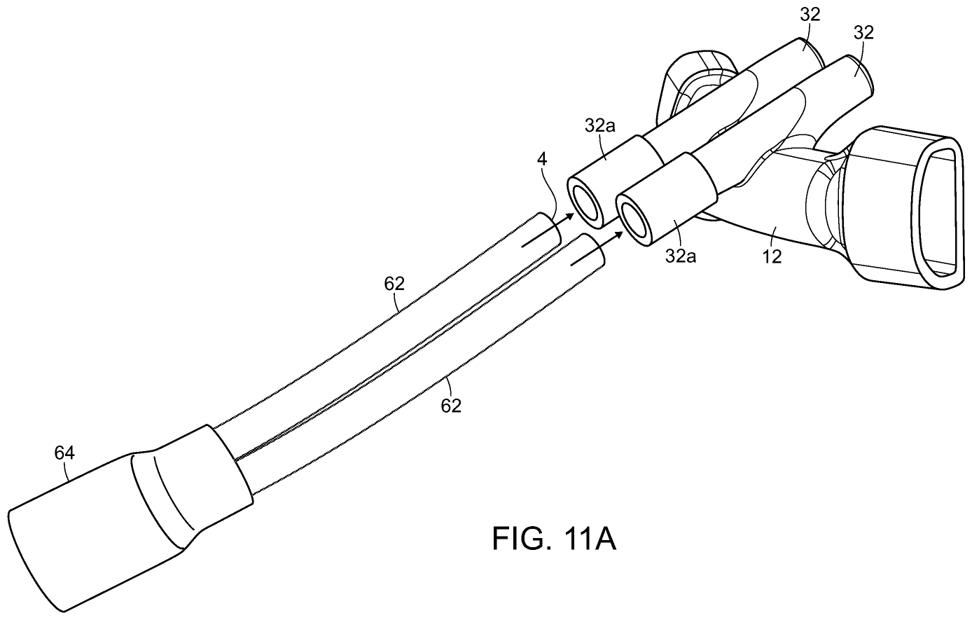

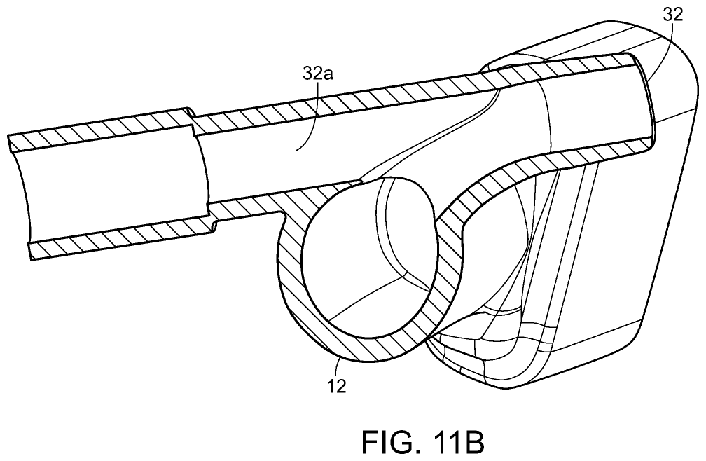

FIG. 11A shows the inlet tubes 62 separate from the parallel ports 32a for purposes of illustration and explanation. The inlet tubes 62 are preferably permanently attached to the parallel ports 32a to provide a secure connection without leaks which may result in a loss or dilution of fluid. FIG. 11B shows the nasal body 12 in cross-section along the nasal prong 32. As shown, the parallel port 32a is fluidly connected to the nasal prongs 32, such that any fluid entering through the inlet tubes 62 is completely mixed with any fluid flowing through the nasal body 12 and the nasal prongs 32.

The ancillary delivery system 60 is preferably used for the introduction of medicines where it is undesirable to remove the nasal cannula system 10 prior to administering the medicine. Removal of the nasal cannula system 10 may be undesirable because of adverse effects from not being connected to the gas supply, or the act of removing and replacing the nasal cannula system 10 may disturb the patient that is otherwise resting. In addition, the ancillary delivery system 60 can be used to administer non-medicinal fluids, such as surfactants or other compounds. Delivery through the nasal cannula system 10 may result in better dispersion and deeper penetration of such medicine or surfactants where such is desired for effectiveness.

FIG. 13 illustrates the inventive nasal cannula system 10 and ancillary delivery system 60 configured in a closed-ended system 52, wherein a single supply adaptor 16 receives both airway tubes 14, connecting both to a single gas supply tube 50. With a single tube 50, both airway tubes are inhalation tubes 14a because both are flowing toward the nasal body 12. Although differently illustrated, the supply adaptor 16 is configured to securely connect the airway tubes 14 to the gas supply tube 50 as described above. The system 10 may also include an air hose 50 connected to the common coupling 64 on the ancillary delivery system 60.

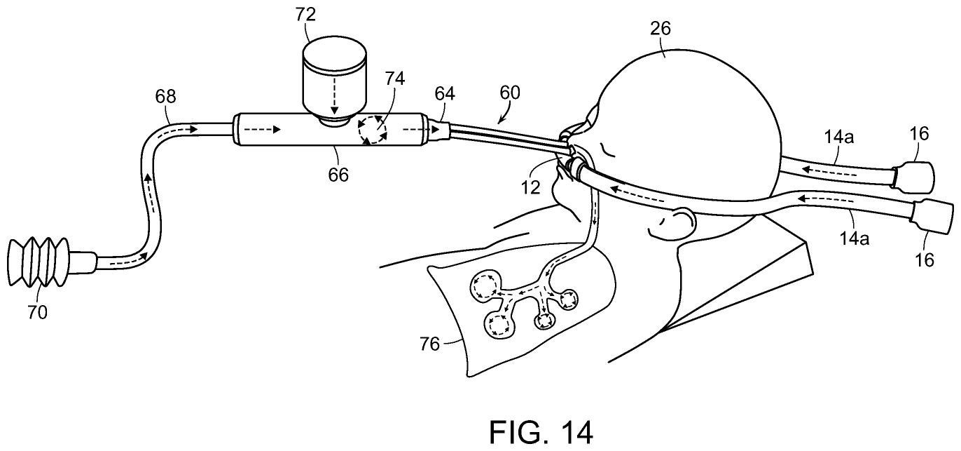

FIG. 14 illustrates an ancillary delivery device 66 configured for connection to the common coupling 64 on the ancillary delivery system 60. The device 66 includes an air hose 68 configured to connect to fluid source 70, such as a pump or other fluid supply, preferably an air tank or other source. The delivery device 66 further includes a hopper 72 that is configured to contain a medicine, surfactant, or other material that is to be administered to a patient. The hopper 72 introduces a predetermined dose of the material, which is directed to an aerosolizer 74, wherein the material is mixed with air or other fluid from the source 70. The aerosolized material is then pushed through the delivery system 60 an introduced to the patient through the parallel ports 32a on the nasal prongs 32. When aerosolized, the material can be more readily forced into the patient's lungs 76 with the air maintaining positive pressure therein. The device 66 may be selectively removed from the delivery system 10 and reattached only when it needs to be used.

The nasal cannula system 10, gas airway tubing 14, and ancillary delivery system 60 described herein has a number of particular features that should preferably be employed in combination. The ancillary delivery system 60 may find utility separately in other nasal cannula systems without departure from the scope and spirit of the invention.

Although preferred embodiments have been described in detail for purposes of illustration, various modifications may be made without departing from the scope and spirit of the invention. Accordingly, the invention is not to be limited, except as by the appended claims.

What is claimed is:

1. A nasal cannula system, comprising:
a nasal body having a nare tube with a nare port configured for insertion into a nasal passage when worn by a patient, wherein the nare tube is in fluid communication with an internal passage in the nasal body;
wherein the nare tube comprises an external parallel port that is in fluid communication with the nare port; and
an ancillary delivery apparatus having an inlet tube connected to the external parallel port.

2. The nasal cannula system of claim 1, wherein the nasal body comprises a pair of nare tubes, both in fluid communication with the internal passage.

3. The nasal cannula system of claim 2, wherein each of the pair of nare tubes comprise an external parallel port.

4. The nasal cannula system of claim 3, wherein the ancillary delivery apparatus comprises a pair of inlet tubes, each of the pair of inlet tubes connected to one of the external parallel ports on the pair of nare tubes.

5. The nasal cannula system of claim 4, further comprising an air hose fluidly connected to the pair of inlet tubes.

6. The nasal cannula system of claim 4, wherein the ancillary delivery apparatus further comprises an aerosolizer for combining an ancillary material with an external air flow.

7. The nasal cannula system of claim 6, further comprising a hopper on the ancillary delivery apparatus for containing the ancillary material and introducing the ancillary material to the aerosolizer.

8. The nasal cannula system of claim 1, further comprising an elongated gas delivery tube communicatingly connected to the nasal body, wherein the gas delivery tube is in fluid communication with the internal passage.

9. The nasal cannula system of claim 8, wherein the gas delivery tube includes a continuous internal rib extending along its length so as to divide the gas delivery tube into two separate passageways.

10. The nasal cannula system of claim 8, wherein the gas delivery tube includes a discontinuous internal rib extending along its length so as to divide the gas delivery tube into two passageways that are in fluid communication through discontinuities in the internal rib.

11. The nasal cannula system of claim 8, wherein the gas delivery tube has a generally flat exterior side configured to lay against a patient's skin when worn.

12. A nasal cannula system, comprising:
a nasal prong having a pair of nare tubes each ending in a nare port configured for insertion into a nasal passage when worn by a patient, wherein the pair of nare tubes are in fluid communication with an internal passage in the nasal prong;
wherein each of the pair of nare tubes comprises an external parallel port that is in fluid communication with the nare port; and
an ancillary delivery apparatus having a pair of inlet tubes, each one connected to the external parallel port on one of the pair of nare tubes.

13. The nasal cannula system of claim 12, further comprising an air hose fluidly connected to the pair of inlet tubes.

14. The nasal cannula system of claim 13, wherein the ancillary delivery apparatus comprises an aerosolizer for combining an ancillary material with an external air flow.

15. The nasal cannula system of claim 14, further comprising a hopper on the ancillary delivery apparatus for containing the ancillary material and introducing the ancillary material to the aerosolizer.

16. The nasal cannula system of claim 12, further comprising an elongated gas delivery tube communicatingly connected to the nasal body, wherein the gas delivery tube is in fluid communication with the internal passage.

17. The nasal cannula system of claim 16, wherein the gas delivery tube includes a continuous internal rib extending along its length so as to divide the gas delivery tube into two separate passageways.

18. The nasal cannula system of claim 16, wherein the gas delivery tube includes a discontinuous internal rib extending along its length so as to divide the gas delivery tube into two passageways that are in fluid communication through discontinuities in the internal rib.

19. The nasal cannula system of claim 16, wherein the gas delivery tube has a generally flat exterior side configured to lay against a patient's skin when worn.
